(19) United States
(12) Patent Application Publication
(10) Pub. No.: US 2025/0186779 A1
(43) Pub. Date: Jun. 12, 2025

(54) SYSTEM AND METHODS TO GUIDE SCS USING SPECIFIC EVOKED POTENTIALS

(71) Applicant: Boston Scientific Neuromodulation Corporation, Valencia, CA (US)

(72) Inventors: Tianhe Zhang, Studio City, CA (US); Jessica Block, Los Angeles, CA (US); Jonathan Philip Larcom, Simi Valley, CA (US); Lisa Denise Moore, Glendale, CA (US); Rafael Carbunaru, Valley Village, CA (US)

(21) Appl. No.: 18/977,452

(22) Filed: Dec. 11, 2024

Related U.S. Application Data
(60) Provisional application No. 63/609,115, filed on Dec. 12, 2023.

(51) Int. Cl. A61N 1/36; A61N 1/372

(52) U.S. Cl. CPC A61N 1/36139; A61N 1/36185; A61N 1/37241; A61N 1/37247

(57) ABSTRACT

This document discusses a neurostimulation device. The neurostimulation device includes a stimulation circuit; a sensing circuit to sense evoked synaptic activity potential (ESAP) signals when coupled to the implantable lead, and a control circuit. The control circuit is configured to initiate delivery of neurostimulation energy by the stimulation circuit, initiate sensing by the sensing circuit of ESAP signals produced by the delivery of neurostimulation energy, change at least one parameter of the neurostimulation energy and continue the sensing of the ESAP signals, compare the sensed ESAP signals to a stored best sensed ESAP signal, wherein the best sensed ESAP signal was previously sensed at a recorded anatomical location using a different trial lead, and determine neurostimulation therapy parameters including a neurostimulation location to produce a best match ESAP signal at the recorded anatomical location that is a closest match to the stored best sensed ESAP signals.

### CLAIM OF PRIORITY

This application claims the benefit of U.S. Provisional Application No. 63/609,115, filed on Dec. 12, 2023, which is hereby incorporated by reference in its entirety.

### TECHNICAL FIELD

This document relates generally to medical devices and more particularly to a system for neurostimulation.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device can be used to program the implantable neurostimulator with stimulation parameters controlling the delivery of the neurostimulation energy.

In one example, the neurostimulation energy is delivered in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Neurostimulation systems may offer many programmable options for the parameters of the neurostimulation to customize the neurostimulation therapy for a specific patient. For some types of neurostimulation (e.g., SCS) the efficacy of the neurostimulation for the patient may depend on an intricate balance of stimulation location coupled with the programmed stimulation waveform. Finding the right stimulation location for a particular patient can be a complicated and time consuming process, and may take a lot of time in the clinic for both the clinic staff and the patient.

### SUMMARY

Electrical neurostimulation energy can be delivered in the form of electrical neurostimulation pulses to treat a neurological condition of the patient. The pulses can be delivered using an implantable stimulation lead. The lead can have multiple electrodes and may be configurable into many electrode configurations. Proper placement of the stimulation lead produces the intended benefit to the patient. Improper lead placement may result in less benefit to the patient and may require a subsequent procedure to reset the neurostimulation and the lead.

Example 1 includes subject matter (such as a neurostimulation device) comprising a stimulation circuit configured to deliver electrical neurostimulation energy to a subject when coupled to an implantable lead, a sensing circuit configured to sense evoked synaptic activity potential (ESAP) signals when coupled to the implantable lead, and a control circuit operatively coupled to the stimulation circuit and the sensing circuit. The control circuit is configured to initiate delivery of neurostimulation energy by the stimulation circuit, initiate sensing by the sensing circuit of ESAP signals produced by the delivery of neurostimulation energy, change at least one parameter of the neurostimulation energy and continue the sensing of the ESAP signals, compare the sensed ESAP signals to a stored best sensed ESAP signal, wherein the best sensed ESAP signal was previously sensed at a recorded anatomical location, and determine neurostimulation therapy parameters including a neurostimulation location to produce a best match ESAP signal at the recorded anatomical location that is a closest match to the stored best sensed ESAP signal.

In Example 2, the subject matter of Example 1 optionally includes a control circuit configured to recurrently change the neurostimulation location for multiple prospective lead placements of the implantable lead, compare the sensed ESAP signals to the stored best sensed ESAP signal, and determine for each prospective lead placement a neurostimulation location that produces the best match ESAP signal at the recorded anatomical location that is a closest match to the stored best sensed ESAP signal.

In Example 3, the subject matter of Example 2 optionally includes a control circuit configured to calculate a correlation of matching of the best match ESAP signal determined for each prospective lead placement to the stored best sensed ESAP signal.

In Example 4, the subject matter of one or both of Examples 2 and 3 optionally includes a control circuit configured to change the neurostimulation location by changing combinations of electrodes of the implantable lead used to deliver the neurostimulation energy, and change the combinations of electrodes of the implantable lead used to sense the ESAP signals in accordance with a change in the electrodes used to deliver the neurostimulation energy.

Example 5 includes subject matter (such as a method of operating a neurostimulation device to deliver electrical neurostimulation therapy) or can optionally be combined with one or any combination of Examples 1-4 to include such subject matter, comprising delivering neurostimulation energy to produce evoked potential signals in a subject using a permanent implantable lead for permanent implant, sensing evoked synaptic activity potential (ESAP) signals using the permanent implantable lead, changing at least one parameter of the neurostimulation energy and continuing to sense the ESAP signals, comparing the sensed ESAP signals to a stored best sensed ESAP signal previously sensed at a recorded anatomical location, and setting neurostimulation therapy parameters to include a neurostimulation location that produces a best match ESAP signal at the recorded anatomical location that is a closest match to the stored best sensed ESAP signal.

In Example 6 the subject matter of Example 5 optionally includes recurrently changing the neurostimulation and comparing the sensed ESAP signals to a stored best sensed ESAP signal for multiple prospective lead placements of the permanent implantable lead, and determining, for each prospective lead placement, a neurostimulation location that produces the best match ESAP signal at the recorded anatomical location that is a closest match to the stored best sensed ESAP signal.

In Example 7, the subject matter of Example 6 optionally includes calculating a correlation of matching of the best match ESAP signal determined for each prospective lead placement to the stored best sensed ESAP signal.

In Example 8, the subject matter of one or any combination of Examples 5-7 optionally includes changing an anatomical location of delivering the neurostimulation energy by changing combinations of electrodes of the permanent implantable lead used to provide the neurostimulation, and changing location of the sensed ESAP signals by changing the combinations of electrodes of the permanent implantable lead used to sense the ESAP signals.

In Example 9, the subject matter of one or any combination of Examples 5-8 optionally includes storing a recording of the best sensed ESAP signal, the anatomical location of the best sensed ESAP signal, and a physiological effect of the trial neurostimulation correlated to the best sensed ESAP signal.

In Example 10, the subject matter of Example 9 optionally includes delivering trial neurostimulation using a stimulation electrode of a trial implantable lead, sensing trial ESAP signals using multiple recording electrode combinations of the trial implantable lead different from the stimulation electrode combination, displaying a representation of the stimulation electrode and the recording electrodes, displaying a metric of the sensed trial ESAP signals in association with the displayed recording electrodes, and receiving a selection of the best sensed ESAP signal by a user interface of the neurostimulation device.

In Example 11, the subject matter of Example 10 optionally includes displaying an indication of anatomical location with the representation of the stimulation electrode and the recording electrodes.

In Example 12, the subject matter of Example 9 optionally includes delivering trial neurostimulation using a stimulation electrode of a trial implantable lead, sensing trial ESAP signals using multiple recording electrodes of the trial implantable lead different from the stimulation electrode, displaying a representation of the stimulation electrode, the recording electrodes, and a representation of one or more physiologic effects in relation to the stimulation electrode and recording electrodes, and receiving a selection of the best sensed ESAP signal by a user interface of the neurostimulation device.

In Example 13, the subject matter of Example 9 optionally includes delivering trial neurostimulation using a stimulation electrode of a trial implantable lead, sensing trial ESAP signals using multiple recording electrodes of the trial implantable lead different from the stimulation electrode, displaying a metric of the sensed trial ESAP signals in association with lead position of a corresponding recording electrode for a sensed trial ESAP signal, displaying the lead position of the stimulation electrode, and receiving a selection of the best sensed ESAP signal by a user interface of the neurostimulation device.

In Example 14, the subject matter of Example 9 optionally includes displaying a representation of anatomical locations available for producing and sensing the trial ESAP signals, and displaying regions of physiologic symptoms in association with the anatomical locations.

Example 15 includes subject matter (such as a neurostimulation device) or can optionally be combined with one or any combination of Examples 1-14 to include such subject matter, comprising a stimulation circuit configured to deliver electrical neurostimulation energy to a subject when coupled to an implantable lead, a sensing circuit configured to sense evoked synaptic activity potential (ESAP) signals when coupled to the implantable lead, and a control circuit operatively coupled to the stimulation circuit and the sensing circuit. The control circuit is configured to initiate delivery of neurostimulation energy by the stimulation circuit, initiate sensing, by the sensing circuit, of ESAP signals produced by the neurostimulation energy, recurrently change neurostimulation location of the delivery of the neurostimulation energy, identify a best sensed ESAP signal determined according to a physiologic effect produced by the neurostimulation energy, and store the best sensed ESAP signal, an anatomical location of sensing of the best sensed ESAP signal, the physiologic effect and the stimulation location associated with the physiological effect.

In Example 16, the subject matter of Example 15 optionally includes a user interface including a display and a control circuit configured to initiate delivery of trial neurostimulation using a stimulation electrode of the implantable lead, initiate sensing of the ESAP signals using multiple recording electrodes of the trial implantable lead different from the stimulation electrode, present, using the display, a representation of the stimulation electrode and the recording electrodes, present a metric of the sensed trial ESAP signals in association with the recording electrodes, and receive a selection of the best sensed ESAP signal via the user interface.

In Example 17, the subject matter of Example 16 optionally includes a control circuit configured to display an indication of anatomical location with the representation of the stimulation electrode and the recording electrodes.

In Example 18, the subject matter of Example 15 optionally includes a user interface including a display and a user input, and control circuit configured to initiate delivery of trial neurostimulation using a stimulation electrode of the implantable lead, initiate sensing of the ESAP signals using multiple recording electrodes of the trial implantable lead different from the stimulation electrode, and present using the display a representation of the stimulation electrode, the recording electrodes, and a representation of one or more physiologic effects in relation to the stimulation electrode and recording electrodes.

In Example 19, the subject matter of Example 15 optionally includes a user interface including a display, and a control circuit configured to initiate delivery of trial neurostimulation using a stimulation electrode of the implantable lead, initiate sensing of the ESAP signals using multiple recording electrodes of the trial implantable lead different from the stimulation electrode, and present, using the display, a metric of the sensed ESAP signals in association with position of one or more of the implantable lead, a stimulation anode, a stimulation cathode, inactive electrodes, a central point of stimulation, and electrode fractionalization.

In Example 20, the subject matter of Example 15 optionally includes a user interface including a display, and a control circuit configured to present, using the display, a representation of anatomical locations available for producing and sensing the ESAP signal and present regions of physiologic symptoms in association with the anatomical locations.

These non-limiting examples can be combined in any permutation or combination. This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of portions of an example of a neurostimulation system.

FIG. 2 is an illustration of portions of another example of a neurostimulation system.

FIG. 3 is an illustration of an example of an implantable pulse generator (IPG) and an implantable lead system.

FIG. 4 is an illustration of another example of an IPG and an implantable lead system.

FIG. 5 is a schematic side view of an example of an electrical stimulation lead.

FIGS. 6A-6H are illustrations of an example of electrodes of a stimulation lead.

FIG. 7 is a block diagram of portions of an example of a neurostimulation device for providing electrical neurostimulation to a patient.

FIG. 8 is a flow diagram of an example of a method to operate a neurostimulation device.

FIG. 9 is an illustration of an example of a user interface display of a neurostimulation device.

FIGS. 10-13 are illustrations of additional examples of a user interface display of a neurostimulation device.

FIG. 14 is a flow diagram of another example of a method to operate a neurostimulation device.

FIGS. 15-18 are illustrations of additional examples of a user interface display of a neurostimulation device.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses devices, systems and methods for programming and delivering electrical neurostimulation to a patient or subject. Advancements in neuroscience and neurostimulation research have led to a demand for delivering complex patterns of neurostimulation energy for various types of therapies. The present system may be implemented using a combination of hardware and software designed to apply any neurostimulation (neuromodulation) therapy, including but not being limited to DBS, SCS, PNS, FES, Occipital Nerve Stimulation (ONS), Sacral Nerve Stimulation (SNS), and Vagus Nerve Stimulation (VNS) therapies.

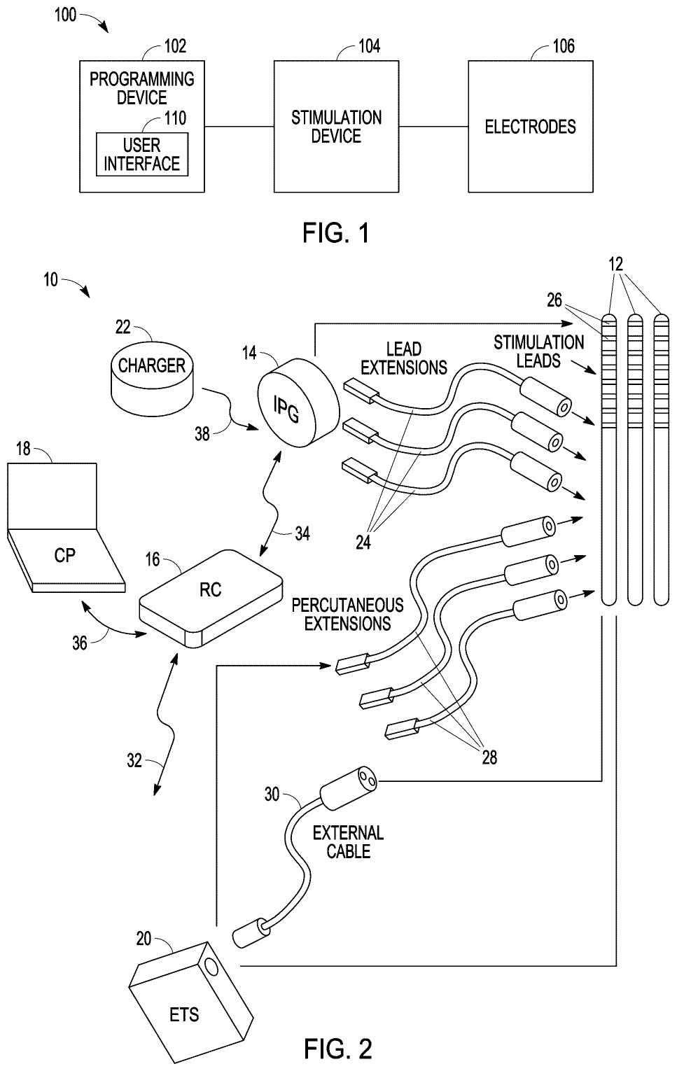

FIG. 1 illustrates an example of portions of a neurostimulation system 100. System 100 includes electrodes 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using multiple stimulation parameters, such as stimulation parameters specifying a pattern of the electrical pulses and a selection of electrodes through which each of the electrical pulses is delivered. In various embodiments, at least some of the stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device 104 via a wired or wireless link.

In this document, a “user” includes a physician or other clinician or caregiver who treats the patient using system 100; a “patient” includes a person who receives or is intended to receive neurostimulation delivered using system 100. In various embodiments, the patient can be allowed to adjust his or her treatment using system 100 to certain extent, such as by adjusting certain therapy parameters and entering feedback and clinical effect information.

In various embodiments, programming device 102 can include a user interface 110 that allows the user to control the operation of system 100 and monitor the performance of system 100 as well as conditions of the patient including responses to the delivery of the neurostimulation. The user can control the operation of system 100 by setting and/or adjusting values of the user-programmable parameters.

In various embodiments, user interface 110 can include a graphical user interface (GUI) that allows the user to set and/or adjust the values of the user-programmable parameters by creating and/or editing graphical representations of various stimulation waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields.

In various embodiments, system 100 can be configured for neurostimulation applications. User interface 110 can be configured to allow the user to control the operation of system 100 for neurostimulation. For example, system 100 as well as user interface 110 can be configured for DBS applications. The DBS configurations include various features that may simplify the task of the user in programming the stimulation device 104 for delivering DBS to the patient, such as the features discussed in this document.

FIG. 2 is an illustration of portions of another example of a neurostimulation system 10 that includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22. The IPG 14 can optionally be physically connected via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The IPG 14 can be implanted into a patient's body, for example, below the patient's clavicle area or within the patient's buttocks or abdominal cavity. The implantable pulse generator can have multiple stimulation channels (e.g., 8 or 16) which may be independently programmable to control the magnitude of the current stimulus from each channel. The IPG 14 can have one, two, three, four, or more connector ports, for receiving the terminals of the leads 12.

The ETS 20 may also be physically connected, optionally via the percutaneous lead extensions 28 and external cable 30, to the stimulation leads 12. The ETS 20, which may have similar pulse generation circuitry as the IPG 14, can also deliver electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a wireless communications link 32. Once the IPG 14 and neurostimulation leads 12 are implanted, the RC 16 may be used to telemetrically communicate with or control the IPG 14 via communications link 34. The communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

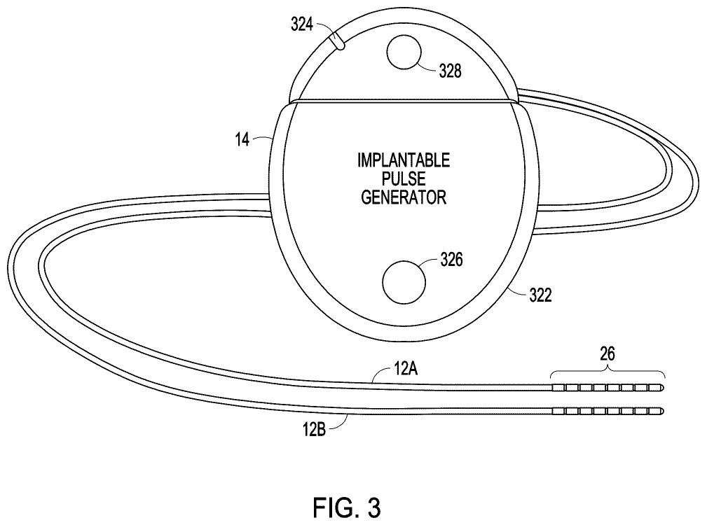

FIG. 3 is an illustration of an example of an IPG 14 (e.g., IPG 14 in FIG. 2) and an implantable lead system that includes stimulation leads (e.g., stimulation leads 12 in FIG. 2). The IPG 14 can be used as stimulation device 104 in FIG. 1. As illustrated in FIG. 3, IPG 14 that can be coupled to implantable leads 12A and 12B at a proximal end of each lead. The distal end of each lead includes electrical contacts or electrodes 26 for contacting a tissue site targeted for electrical neurostimulation. As illustrated in FIG. 3, leads 12A and 12B each include 8 electrodes 26 at the distal end. The number and arrangement of leads 12A and 12B and electrodes 26 as shown in FIGS. 2 and 3 are only examples, and other numbers and arrangements are possible. In various examples, the lead electrodes 26 are ring electrodes. In various examples the lead electrodes 26 include one or more segmented electrodes.

The IPG 14 can include a hermetically sealed IPG case 322 to house the electronic circuitry of IPG 14. IPG 14 can include an electrode 326 formed on IPG case 322. IPG 14 can include an IPG header 324 for coupling the proximal ends of leads 12A and 12B. IPG header 324 may optionally also include an electrode 328. One or both of electrodes 326 and 328 may be used as a reference electrode.

The implantable leads and electrodes may be configured by shape and size to provide electrical neurostimulation energy to a neuronal target included in the subject's brain. Neurostimulation energy can be delivered in a monopolar (also referred to as unipolar) mode using electrode 326 or electrode 328 and one or more electrodes selected from electrodes 26. Neurostimulation energy can be delivered in a bipolar mode using a pair of electrodes of the same lead (lead 12A or lead 12B). Neurostimulation energy can be delivered in an extended bipolar mode using one or more electrodes of a lead (e.g., one or more electrodes of lead 12A) and one or more electrodes of a different lead (e.g., one or more electrodes of lead 12B).

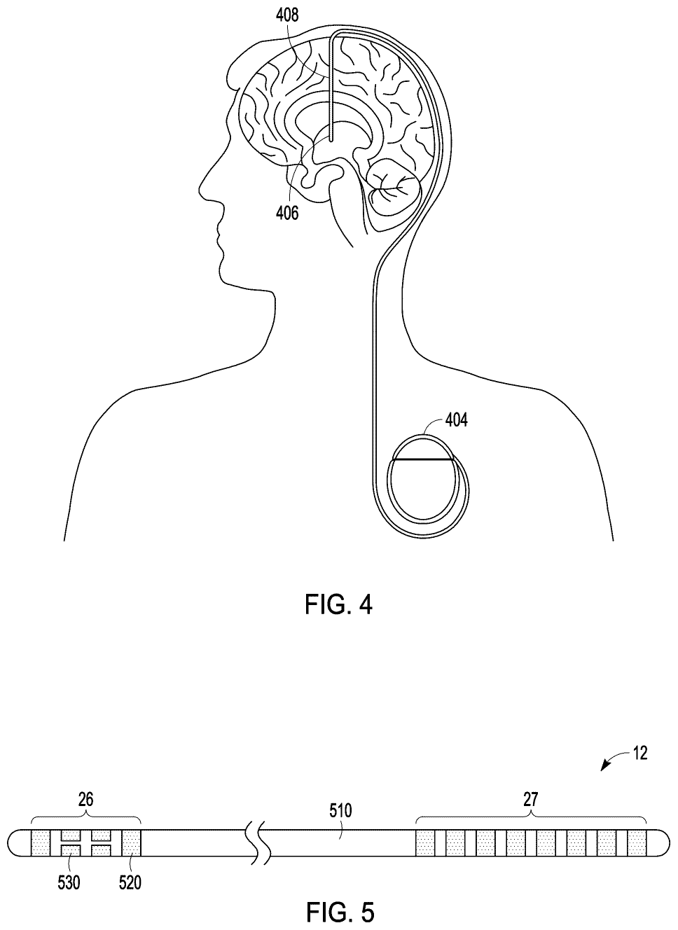

FIG. 4 illustrates another example of an IPG 404 and an implantable lead system 408 arranged to provide neurostimulation to a patient. An example of IPG 404 includes IPG 14 of FIGS. 2 and 3. An example of lead system 408 includes one or more of leads 12A and 12B in FIG. 3. The distal end 406 of the lead includes multiple electrodes (e.g., electrodes 26 in FIG. 3). In the illustrated embodiment, implantable lead system 408 is arranged to provide Deep Brain Stimulation (DBS) to a patient, with the stimulation target being neuronal tissue in a subdivision of the thalamus of the patient's brain. Other examples of DBS targets include neuronal tissue of the globus pallidus (GPi), the subthalamic nucleus (STN), the pedunculopontine nucleus (PPN), substantia nigra pars reticulate (SNr), cortex, globus pallidus externus (GPe), medial forebrain bundle (MFB), periaquaductal gray (PAG), periventricular gray (PVG), habenula, subgenual cingulate, ventral intermediate nucleus (VIM), anterior nucleus (AN), other nuclei of the thalamus, zona incerta, ventral capsule, ventral striatum, nucleus accumbens, and any white matter tracts connecting these and other structures.

After implantation, a clinician will program the neurostimulation device 400 using a CP 18, remote control, or other programming device. The programmed neurostimulation device 400 can be used to treat a neurological condition of the patient, such as Parkinson's Disease, Tremor, Epilepsia, Alzheimer's Disease, other Dementias, Stroke, Multiple Sclerosis, Amyotrophic Lateral Sclerosis (ALS), Autism, brain injury, brain tumor, migraine or other pain or headache condition, and any neurological syndromes that are congenic, degenerative, or acquired.

Returning to FIG. 3, the electronic circuitry of IPG 14 can include a stimulation control circuit that controls delivery of the neurostimulation energy. The stimulation control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

FIG. 5 is a schematic side view of an embodiment of an electrical stimulation lead. FIG. 5 illustrates a stimulation lead 12 with electrodes 26 disposed at least partially about a circumference of the lead 12 along a distal end portion of the lead and terminals 27 disposed along a proximal end portion of the lead. The lead 12 can be implanted near or within the desired portion of the body to be stimulated (e.g., the brain, spinal cord, or other body organs or tissues). In one example of operation for deep brain stimulation, access to the desired position in the brain can be accomplished by drilling a hole in the patient's skull or cranium with a cranial drill (commonly referred to as a burr), and coagulating and incising the dura mater, or brain covering. The lead 12 can be inserted into the cranium and brain tissue with the assistance of a stylet (not shown). The lead 12 can be guided to the target location within the brain using, for example, a stereotactic frame and a microdrive motor system. In some embodiments, the microdrive motor system can be fully or partially automatic. The microdrive motor system may be configured to perform one or more the following actions (alone or in combination): insert the lead 12, advance the lead 12, retract the lead 12, or rotate the lead 12.

In some embodiments, measurement devices coupled to the muscles or other tissues stimulated by the target neurons, or a unit responsive to the patient or clinician, can be coupled to the implantable pulse generator or microdrive motor system. The measurement device, user, or clinician can indicate a response by the target muscles or other tissues to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

The lead 12 for deep brain stimulation can include stimulation electrodes, recording electrodes, or both. In at least some embodiments, the lead 12 is rotatable so that the stimulation electrodes can be aligned with the target neurons after the neurons have been located using the recording electrodes. Stimulation electrodes may be disposed on the circumference of the lead 12 to stimulate the target neurons. Stimulation electrodes may be ring-shaped so that current projects from each electrode equally in every direction from the position of the electrode along a length of the lead 12. In the embodiment of FIG. 5, two of the electrodes 520 are ring electrodes 520. Ring electrodes typically do not enable stimulus current to be directed from only a limited angular range around of the lead. Segmented electrodes 530, however, can be used to direct stimulus current to a selected angular range around the lead. When segmented electrodes 530 are used in conjunction with an IPG 14 that delivers constant current stimulus, current steering can be achieved to more precisely deliver the stimulus to a position around an axis of the lead (e.g., radial positioning around the axis of the lead). To achieve current steering, segmented electrodes can be utilized in addition to, or as an alternative to, ring electrodes.

The lead 12 includes a lead body 510, terminals 27, and one or more ring electrodes 520 and one or more sets of segmented electrodes 530 (or any other combination of electrodes). The lead body 510 can be formed of a biocompatible, non-conducting material such as, for example, a polymeric material. Suitable polymeric materials include, but are not limited to, silicone, polyurethane, polyurea, polyurethaneurea, polyethylene, or the like. Once implanted in the body, the lead 12 may be in contact with body tissue for extended periods of time. In at least some embodiments, the lead 12 has a cross-sectional diameter of no more than 1.5 millimeters (1.5 mm) and may be in the range of 0.5 to 1.5 mm. In at least some embodiments, the lead 12 has a length of at least 10 centimeters (10 cm) and the length of the lead 12 may be in the range of 10 to 70 cm.

The electrodes 26 can be made using a metal, alloy, conductive oxide, or any other suitable conductive biocompatible material. Examples of suitable materials include, but are not limited to, platinum, platinum iridium alloy, iridium, titanium, tungsten, palladium, palladium rhodium, or the like. Preferably, the electrodes are made of a material that is biocompatible and does not substantially corrode under expected operating conditions in the operating environment for the expected duration of use. Each of the electrodes can either be used or unused (OFF). When the electrode is used, the electrode can be used as an anode or cathode and carry anodic or cathodic current. In some instances, an electrode might be an anode for a period of time and a cathode for a period of time.

Deep brain stimulation leads and other leads may include one or more sets of segmented electrodes. Segmented electrodes may provide for superior current steering than ring electrodes because target structures in deep brain stimulation or other stimulation are not typically symmetric about the axis of the distal electrode array. Instead, a target may be located on one side of a plane running through the axis of the lead. Through the use of a radially segmented electrode array (“RSEA”), current steering can be performed not only along a length of the lead but also around a circumference of the lead. This provides precise three-dimensional targeting and delivery of the current stimulus to neural target tissue, while potentially avoiding stimulation of other tissue.

Any number of segmented electrodes 530 may be disposed on the lead body 510 including, for example, anywhere from one to sixteen or more segmented electrodes 530. It will be understood that any number of segmented electrodes 530 may be disposed along the length of the lead body 510. A segmented electrode 530 typically extends only 75%, 67%, 60%, 50%, 40%, 33%, 25%, 20%, 17%, 15%, or less around the circumference of the lead.

The segmented electrodes 530 may be grouped into sets of segmented electrodes, where each set is disposed around a circumference of the lead 12 at a particular longitudinal portion of the lead 12. The lead 12 may have any number segmented electrodes 530 in a given set of segmented electrodes. The lead 12 may have one, two, three, four, five, six, seven, eight, or more segmented electrodes 530 in a given set. In at least some embodiments, each set of segmented electrodes 530 of the lead 12 contains the same number of segmented electrodes 530. The segmented electrodes 530 disposed on the lead 12 may include a different number of electrodes than at least one other set of segmented electrodes 530 disposed on the lead 12. The segmented electrodes 530 may vary in size and shape. In some embodiments, the segmented electrodes 530 are all of the same size, shape, diameter, width or area or any combination thereof. In some embodiments, the segmented electrodes 530 of each circumferential set (or even all segmented electrodes disposed on the lead 12) may be identical in size and shape.

Each set of segmented electrodes 530 may be disposed around the circumference of the lead body 510 to form a substantially cylindrical shape around the lead body 510. The spacing between individual electrodes of a given set of the segmented electrodes may be the same, or different from, the spacing between individual electrodes of another set of segmented electrodes on the lead 12. In at least some embodiments, equal spaces, gaps or cutouts are disposed between each segmented electrode 530 around the circumference of the lead body 510. In other embodiments, the spaces, gaps or cutouts between the segmented electrodes 530 may differ in size, or cutouts between segmented electrodes 530 may be uniform for a particular set of the segmented electrodes 530 or for all sets of the segmented electrodes 530. The sets of segmented electrodes 530 may be positioned in irregular or regular intervals along a length the lead body 510.

Conductor wires (not shown) that attach to the ring electrodes 520 or segmented electrodes 530 extend along the lead body 510. These conductor wires may extend through the material of the lead 12 or along one or more lumens defined by the lead 12, or both. The conductor wires couple the electrodes 520, 530 to the terminals 27.

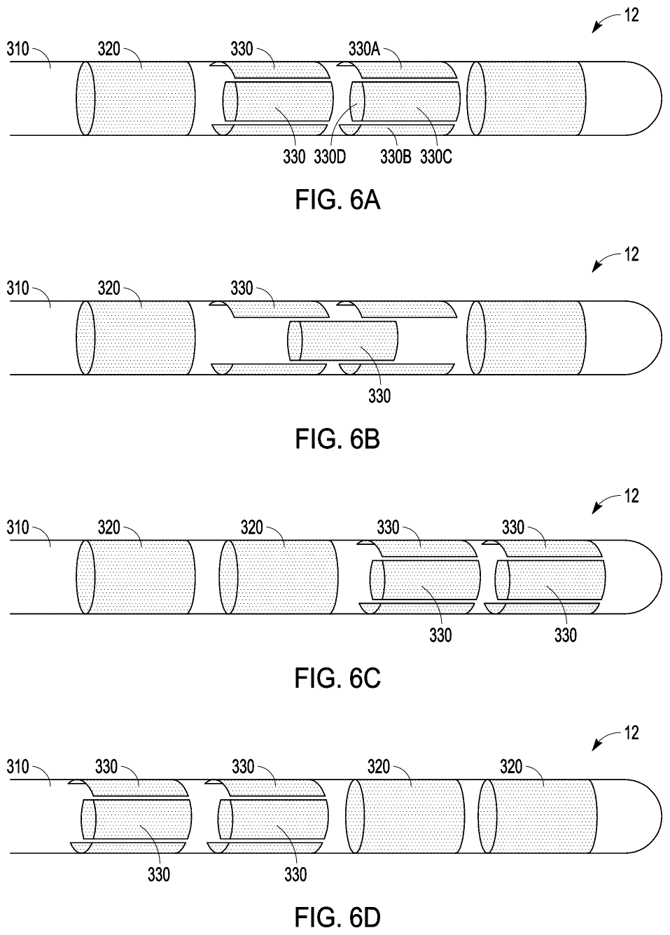

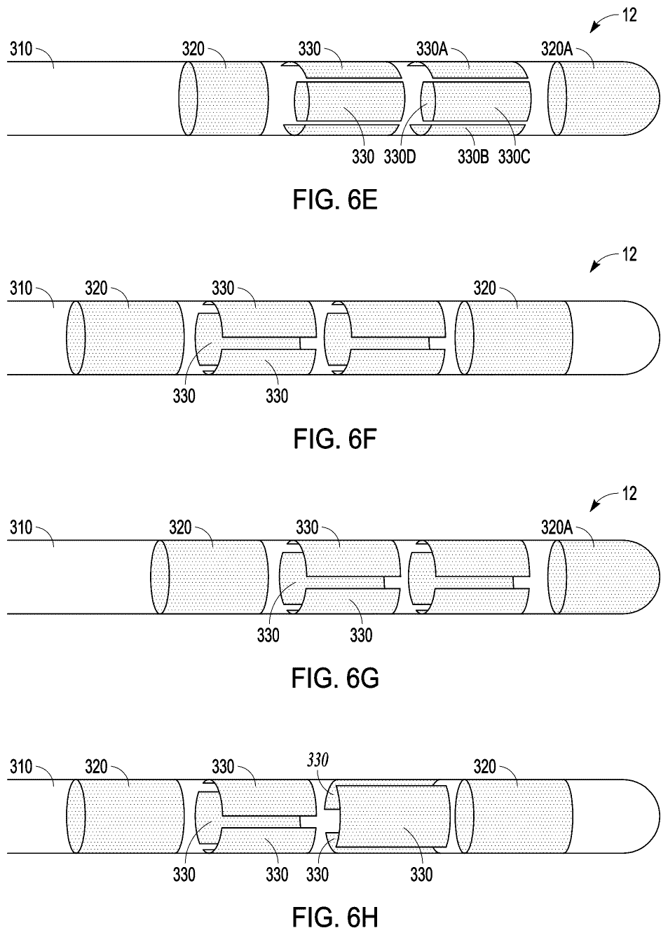

FIGS. 6A-6H are illustrations of different embodiments of leads 12 with segmented electrodes 330, optional ring electrodes 320 or tip electrodes 320a, and a lead body 310. The sets of segmented electrodes 330 each include either two (FIG. 6B), three (FIGS. 6E-6H), or four (FIGS. 6A, 6C, and 6D) or any other number of segmented electrodes including, for example, three, five, six, or more. The sets of segmented electrodes 330 can be aligned with each other (FIGS. 6A-6G) or staggered (FIG. 6H).

When the lead 12 includes both ring electrodes 320 and segmented electrodes 330, the ring electrodes 320 and the segmented electrodes 330 may be arranged in any suitable configuration. For example, when the lead 12 includes two ring electrodes 320 and two sets of segmented electrodes 330, the ring electrodes 120 can flank the two sets of segmented electrodes 330 (see e.g., FIGS. 5, 6A, and 6E-6H, ring electrodes 320 and segmented electrode 330). Alternately, the two sets of ring electrodes 320 can be disposed proximal to the two sets of segmented electrodes 330 (see e.g., FIG. 6C, ring electrodes 320 and segmented electrode 330), or the two sets of ring electrodes 320 can be disposed distal to the two sets of segmented electrodes 330 (see e.g., FIG. 6D, ring electrodes 320 and segmented electrode 330). One of the ring electrodes can be a tip electrode (see e.g., tip electrode 320a of FIGS. 6E and 6G). It will be understood that other configurations are possible as well (e.g., alternating ring and segmented electrodes, or the like).

By varying the location of the segmented electrodes 330, different coverage of the target neurons may be selected. For example, the electrode arrangement of FIG. 6C may be useful if the physician anticipates that the neural target will be closer to a distal tip of the lead body 310, while the electrode arrangement of FIG. 6D may be useful if the physician anticipates that the neural target will be closer to a proximal end of the lead body 310.

Any combination of ring electrodes 320 and segmented electrodes 330 may be disposed on the lead 12. For example, the lead 12 may include a first ring electrode 320, two sets of segmented electrodes; each set formed of four segmented electrodes 330, and a final ring electrode 320 at the end of the lead. This configuration may simply be referred to as a 1-4-4-1 configuration (FIGS. 6A and 6E, ring electrodes 320 and segmented electrode 330). It may be useful to refer to the electrodes with this shorthand notation. Thus, the embodiment of FIG. 6C may be referred to as a 1-1-4-4 configuration, while the embodiment of FIG. 6D may be referred to as a 4-4-1-1 configuration. The embodiments of FIGS. 6F, 6G, and 6H can be referred to as a 1-3-3-1 configuration. Other electrode configurations include, for example, a 2-2-2-2 configuration, where four sets of segmented electrodes are disposed on the lead, and a 4-4 configuration, where two sets of segmented electrodes, each having four segmented electrodes 330 are disposed on the lead. The 1-3-3-1 electrode configuration of FIGS. 6F, 6G, and 6H has two sets of segmented electrodes, each set containing three electrodes disposed around the circumference of the lead, flanked by two ring electrodes (FIGS. 6F and 6H) or a ring electrode and a tip electrode (FIG. 6G). In some embodiments, the lead includes 16 electrodes. Possible configurations for a 16-electrode lead include but are not limited to 4-4-4-4; 8-8; 3-3-3-3-3-1 (and all rearrangements of this configuration); and 2-2-2-2-2-2-2-2.

Any other suitable arrangements of segmented and/or ring electrodes can be used. As an example, arrangements in which segmented electrodes are arranged helically with respect to each other. One embodiment includes a double helix. One or more electrical stimulation leads can be implanted in the body of a patient (for example, in the brain or spinal cord of the patient) and used to stimulate surrounding tissue. The lead(s) are coupled to the implantable pulse generator (such as IPG 14 in FIG. 2).

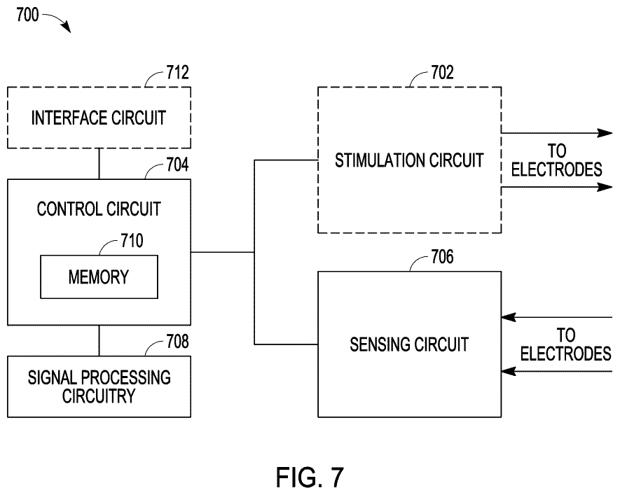

FIG. 7 is a block diagram of portions of an embodiment of a neurostimulation device 700 for providing neurostimulation. The neurostimulation device 700 may be an ETS (e.g., ETS 20 in FIG. 1) or other type of external stimulator. The neurostimulation device 700 includes a stimulation circuit 702, a control circuit 704, and a sensing circuit 706. The stimulation circuit 702 can be operatively coupled to stimulation electrodes such as any of the electrodes described herein and the stimulation circuit 702 provides or delivers electrical neurostimulation energy to the electrodes. The control circuit 704 can include a processor such as a microprocessor, a digital signal processor, application specific integrated circuit (ASIC), or other type of processor, interpreting or executing instructions in software modules or firmware modules. The instructions can be stored in memory 710 that can be integral to the control circuit 704 or separate from the control circuit 704. The control circuit 704 can include other circuits or sub-circuits to perform the functions described. These circuits may include software, hardware, firmware, or any combination thereof. Multiple functions can be performed in one or more of the circuits or sub-circuits as desired.

The neurostimulation device 700 includes a sensing circuit 706. An example of the sensing circuit 706 includes one or more sense amplifiers switchable among recording electrodes to sense internal neural signals of the patient. The neurostimulation device 700 may include signal processing circuitry 708. The signal processing circuitry 708 can include one or more processes running on a processor to perform signal analysis or other signal processing on the neural signals sensed using the sensing circuit 706.

The neurostimulation device 700 is connectable to at least one stimulation lead (e.g., stimulation lead 12 in FIG. 5) that can be implanted in the body of a patient (for example, in the brain or spinal cord of the patient) and the electrodes of the stimulation leads are used to stimulate surrounding tissue. The neurostimulation device 700 can be used to evaluate the placement of stimulation leads. The stimulation circuit 702, sensing circuit 706, control circuit 704, and signal processing circuitry 708 can be included in the same device of the system to evaluate the lead position. The neurostimulation device 700 can include a user interface 712. The user interface 712 may include one or more of a display, a mouse, a keyboard, and a touch sensitive or multi-touch sensitive display screen. In some examples, the neurostimulation device 700 can be used some time after implant to evaluate the efficacy of the electrode configuration, or aid in producing efficacious neurostimulator settings.

When appropriately placed, the stimulation lead 12 provides stimulation energy (e.g., electrical current) to the tissue of the neurostimulation target and the stimulation produces the intended benefit to the patient with a minimum of side effects. Proper lead placement may also lead to lower energy needed to produce the desired result. When the lead is not properly placed, the neurostimulation may result in one or both of lack of therapy and unintended side effects.

The human nervous system produces a neural response to neurostimulation received via sensory receptors or received directly into any part of the network of neural elements that forms the nervous system. Additionally, neurostimulation can excite elements of the human nervous system in a manner that produces neural responses. These neural responses are known as evoked potentials. Evoked potential (EP) signals can be sensed electrically by the neurostimulation device 700, such as by using sense amplifiers of the sensing circuit 706 coupled to recording electrodes for example. A repetitive stimulus can be applied or presented to the nervous system and the evoked potential signals (e.g., evoked resonant neural activity (ERNA) signals) sensed from the presentations can be processed (e.g., filtered by averaging) to detect presence of evoked potentials.

Changes in the sensed evoked potential signals can provide information regarding the efficacy of placement of the stimulation lead. The way in which the evoked potential signals change in response to one or more of changes in the number of presentations of the stimulation, changes in timing or conditions of the stimulation, and changes in the properties of the stimulation can provide information of the proximity (e.g., distance and direction) of the stimulation lead to the preferred lead location for the neurostimulation. The preferred lead location for the neurostimulation may be a target tissue volume, or sub-volume, a preferred stimulation location, a stimulation location where evoking stimulation energy affect electrophysiology (EP) responses in the desired manner (e.g., by maximizing a particular EP response feature), or a location where stimulation produces the desired response (e.g., maximizing a therapeutic effect, counteracting or minimizing a side effect, etc.).

Neurostimulation (neuromodulation) therapy such as SCS may involve a temporary “trial phase” in which neurostimulation is provided using a neurostimulation device such as an ETS and a temporary or trial implantable lead. Neurostimulation parameters are tested and evaluated, and the best lead placement is determined. After information is collected for the trial phase, the trial lead is explanted. This is followed sometime later (e.g., a few weeks later) by a more permanent procedure in which an implantable lead for permanent use is implanted and attached to an IPG (e.g., IPG 14 in FIG. 1).

In the more permanent procedure, the permanent lead is implanted but reconciling the previous trial lead placement and settings with the new permanent lead and IPG settings to get the same patient benefit as the trial can be complicated. Typically, fluoroscopic imaging is used to place the permanent lead position in the same position as the trial lead, but imaging alone may not enable full reconciliation between the permanent placement and the temporary placement.

An Evoked Compound Activity Potential (ECAP) is a type of evoked potential that is a measure of the electrical response from tissue to electrical neurostimulation. ECAP is a compound response generated by a group of electrically activated nerve fibers. An Evoked Synaptic Activity Potential (ESAP) is a type of evoked potential that is detectable epidurally. Unlike ECAPs, ESAPs are placement sensitive, do not propagate, and are block-able by neurotransmitter antagonists. ESAPs potentially represent the activation of specific gray matter elements (i.e., superficial elements that receive AMPA synapses from afferents) that are specific to, and may be concordant with, placement-specific physiological effects rather than a broadly propagating response generated by a group of nerve fibers such as a dorsal column or axonal elements.

Because ESAPs are location specific, ESAP signals can be measured during trial lead placement and permanent lead placement (e.g., using an external neurostimulation device). The ESAP measurements can aid in reconciling trial and permanent implants to enable consistent therapy between implants. For example, ESAPs can be sensed during a trial neurostimulation. Physiological effects at stimulation sites where stimulation worked to produce a desired response can be correlated to sensed ESAP signals produced by the stimulation. The position of an ESAP signal can be recorded with respect to lead position or vertebrate location. The anatomical correlation between the sensed ESAP signal and the physiologic effect can be recorded and used for guidance during the later permanent implant procedure.

During the permanent implant procedure, the location at which the ESAP signal was observed versus the best patient outcome can be presented to the physician. The patient outcomes for the procedures may be pain-related outcomes, autonomic outcomes (e.g., outcomes related to blood flow, visceral signals, blood pressure, cardiac signals, etc.), movement and motor related outcomes, side effect related outcomes, etc. The physician can use the presented localization of the ESAP to guide placement of the permanent lead and provide automatic programming of the IPG to be connected to the permanent lead.

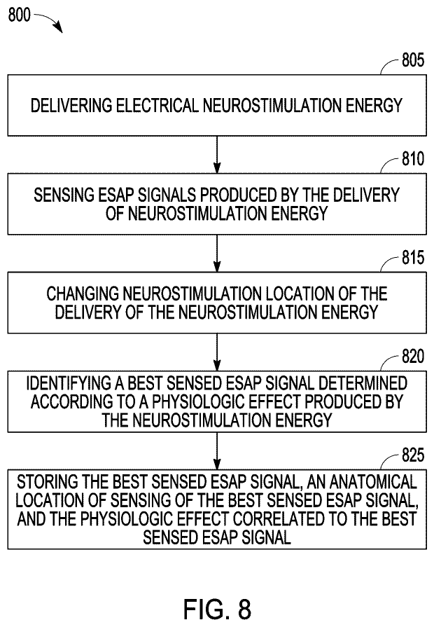

FIG. 8 is a flow diagram of an example of a method 800 of operating a neurostimulation device. The neurostimulation device may be an ETS (e.g., the ETS 20 of FIG. 1) connected to a trial implantable lead, and the method 800 collects ESAP information using a trial stimulation procedure. The ESAP information is used in a later permanent lead implant procedure.

At block 805, the control circuit of the neurostimulation device (e.g., control circuit 704 of the neurostimulation device 700 in the example of FIG. 7) initiates delivery of neurostimulation energy by the stimulation circuit of the device (e.g., stimulation circuit 702 of the device 700 in the example of FIG. 7). At block 810, the control circuit initiates sensing of ESAP signals using the sensing circuit of the device (e.g., sensing circuit 706 of the device 700 in the example of FIG. 7). The ESAP signals are produced by the delivery of neurostimulation energy.

At block 815, the control circuit performs a process or algorithm that recurrently changes parameters of the neurostimulation including the anatomical location of the neurostimulation. The trial lead includes multiple electrodes, such as any of the multi-electrode leads described herein. To change location of the neurostimulation, the control circuit changes combinations of electrodes of the trial implantable lead used to provide the neurostimulation. Other electrodes are used to sense the ESAP signal response to the neurostimulation. For instance, the control circuit may perform a sweep of the electrodes of the trial lead in which most or all the available electrode stimulation combinations are used in turn for stimulation while other electrodes are used to sense the response to the stimulation.

At block 820, the best sensed ESAP signal produced by the neurostimulation is identified. The best ESAP signal may be identified using signal processing circuitry of the neurostimulation device. The best ESAP signal may be the ESAP signal with a desired signal feature. The best ESAP signal produced by the neurostimulation is correlated to the physiologic effect produced by the neurostimulation.

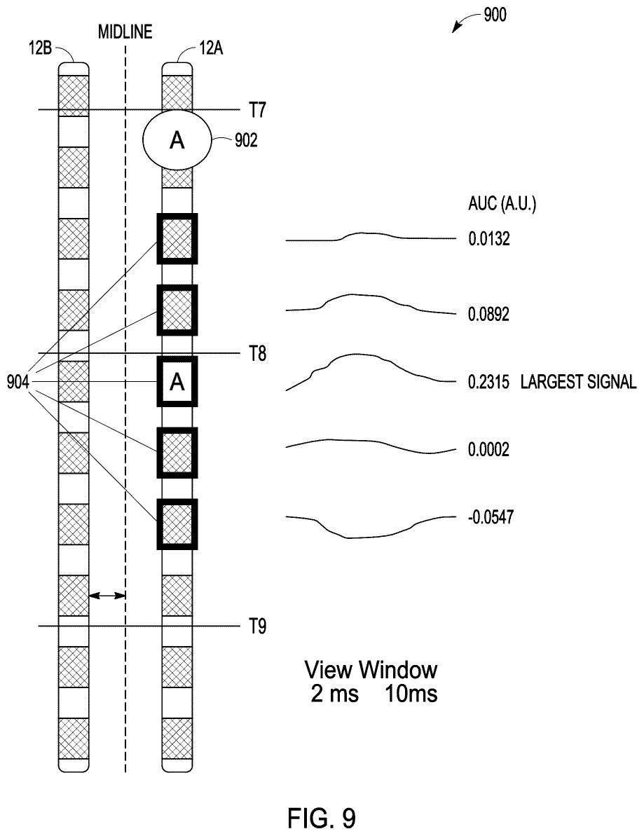

FIG. 9 is an illustration of an example of a user interface display of a neurostimulation device showing a mapping of trial stimulation to ESAP signal recording. The user interface may be the user interface of a neurostimulation device used in a trial stimulation procedure. The display 900 includes a representation of two trial leads 12A and 12B, and shows stimulation location 902 on lead 12A. The stimulation location 902 may represent the central point of stimulation (CPS), or another stimulation location (e.g., a fractionalization of the stimulation, the cathode with the strongest stimulation, etc.). The stimulation location 902 can indicate the stimulation electrode used or a stimulation electrode combination. The stimulation location 902 may be associated with a lead placement to achieve a specific patient outcome for SCS (e.g., lower blood pressure, angina pain relief, etc.).

The display 900 shows recording electrodes 904 used to sense one or more ESAP signals resulting from the stimulation. The control circuit may calculate one or more metrics of the sensed ESAP signals and present the metrics on the display. In the example of FIG. 9, waveforms of the ESAP signals are displayed next to the recording electrodes along with the area under the curve (AUC) of the waveforms. Other examples of the metric that be determined include any ESAP signal magnitude, height of the first peak of the ESAP signal, time domain property, frequency domain property (e.g., peak height), area under the curve, curve length, peak latency, etc. The “View Window” field of the display 900 shows the displayed waveforms are for a viewing window from 2 milliseconds (2 ms) to 10 ms after the recording begins. The waveforms and metric can provide a profile of ESAP signals along the lead 12A. The display 900 may also show an anatomical location with the lead electrodes. In the example of FIG. 9, the display 900 shows a representation of the T7, T8, and T9 vertebra relative to the electrodes of the leads 12A, 12B. In some examples, display can be presented as overlay on an anatomical image (e.g., an anatomical image of the T7, T8, and T9 vertebra).

The neurostimulation and sensing can be repeated with different stimulation locations and other stimulation parameters (e.g., different stimulation amplitudes). This can result in multiple profiles of sensed ESAP signals along the lead 12A for different stimulation configurations.

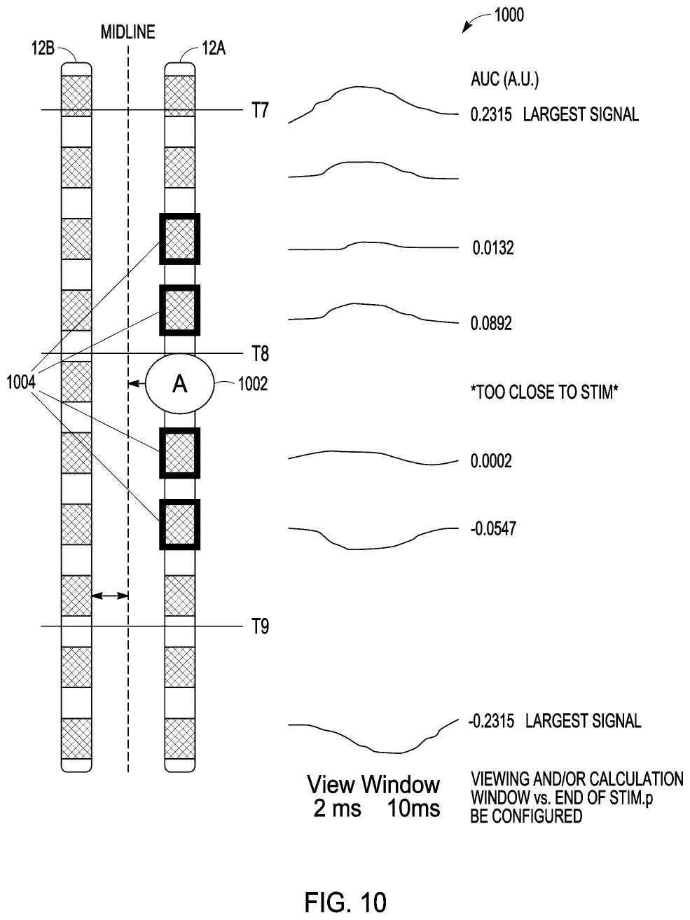

FIG. 10 is an illustration of another example of a user interface display of a neurostimulation device showing a mapping of trial stimulation to ESAP signal recording. The display 1000 is similar to the example of FIG. 9. In the example of FIG. 10, the stimulation point 1002 is at a different electrode location of lead 12A. The recording electrodes 1004 used may exclude the electrode used for the simulation or an electrode that is too close to the stimulation location. The stimulation and recording process can be repeated for stimulation lead 12B or the process can include stimulation and sensing using both leads 12A and 12B. The leads can be oriented rostrocaudally or mediolaterally. The electrodes can be configured for differential, single-ended, referential, or other recording geometries. Whatever recording geometry is used can be displayed.

The user of the neurostimulation device can review the recorded profiles of ESAP signals on the display and may enter a selection of the best sensed ESAP signal. In certain examples, the control circuit may present a recommendation of the best sensed ESAP signal, and the user acknowledges the ESAP signal as the best ESAP signal or makes their own selection of the best ESAP signal. In some examples, the control circuit may select the recommended best sensed ESAP signal according to a rule-based algorithm.

Returning to FIG. 8 at block 825, the best sensed ESAP signal is stored by the neurostimulation device or sent to another device for storing and also the anatomical location of sensing of the best sensed ESAP signal. The best sensed ESAP signal and location can be referenced during placement of the permanent lead in the future procedure. Recreation of the best sensed ESAP signal at the desired location would indicate best placement of the permanent lead. The lead stimulation and sensing profiles can be stored and tied to a physiological effect of the neurostimulation and the best sensed ESAP signal can be correlated to a physiological effect.

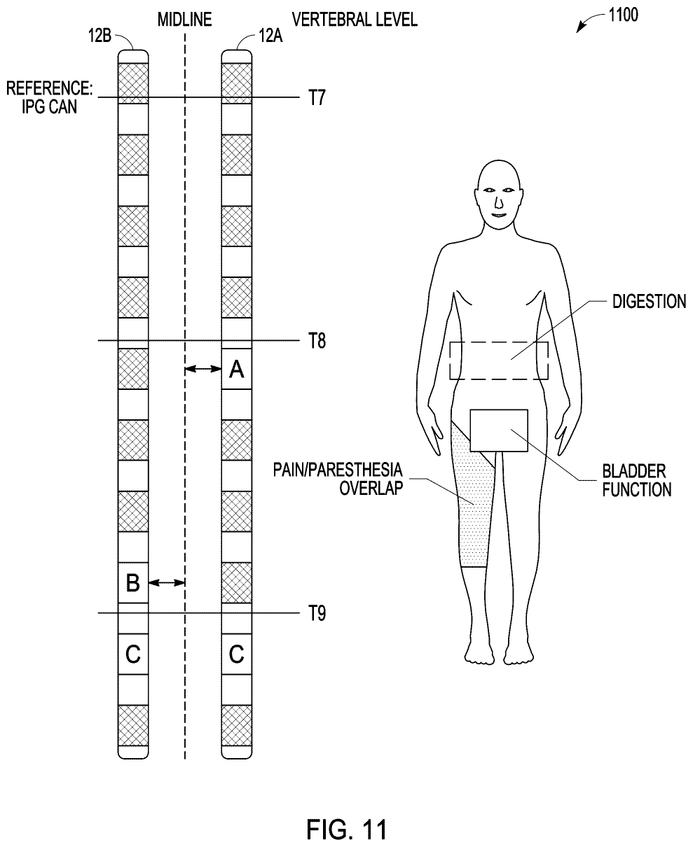

FIG. 11 is an illustration of another example of a user interface display of a neurostimulation device. The display 1100 shows a mapping of physiological measures versus the sensed ESAP center for a trial stimulation procedure. The diagram in the example of FIG. 11 shows the relationship between location of the sensed ESAP resulting from neurostimulation and the physiologic effect of the stimulation. The lead electrodes labeled A, B, and C denote the lead electrodes and lead location where the ESAP signal was recorded. The display 1100 also includes the physiological effects of digestion associated with electrode A, pain and paresthesia overlap associated with electrode B, and bladder function associated with electrodes C. The physiological effects can be displayed as text or graphically using the model anatomy. The physiological effects can be associated to the electrodes on the display using shading or color or other method.

Other examples of physiological effect that can be included in a stimulation and response profile include behavioral responses, effects reported in a patient survey, pain relief, cardiac effects (e.g., changes in heart rate), blood flow or peripheral effect (measurable via infrared camera, galvanic skin response, etc.), autonomic-related visceral symptoms (digestion, bladder control, bowel movement, sexual function, etc.), and motor responses (patient report movement enhancement, electromyography results, proprioception, etc.). In conjunction with a serum draw, the physiological effect can be autonomic-related systemic or serum-detectable events (e.g., concentrations of pro/anti-inflammatory interleukins and cytokines, hormonal concentrations, insulin concentrations, glucose concentrations, etc.).

The physiological effect and the electrode location can be included in metadata stored with the recorded ESAP signals. The metadata can also include measures of distance versus midline of the locations, markers denoting vertebra rostrocaudal position, which can be useful for alignment purposes during the permanent implant procedure. The physiological data and ESAP map data may be saved in a report generated for the trial stimulation procedure.

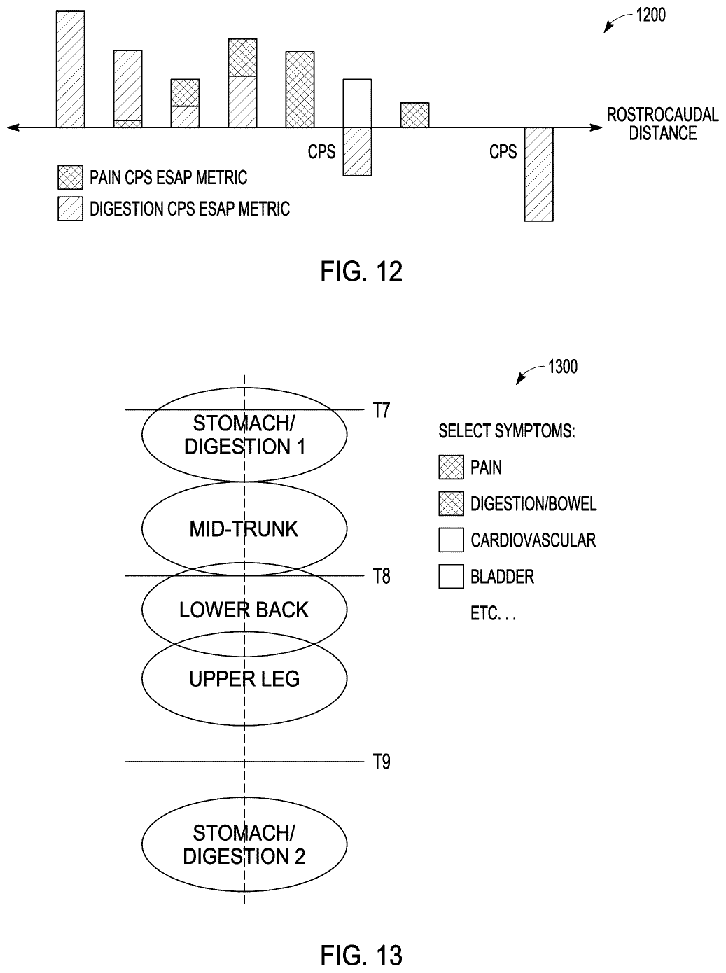

FIG. 12 is an illustration of another example of a user interface display of a neurostimulation device. The display 1200 shows a horizontal representation of the electrodes of an implantable lead with the vertical bars corresponding to recording electrode locations. The height of the bars corresponds to an ESAP signal characteristic or metric of sensed ESAP signals resulting from neurostimulation applied by the neurostimulation device. The control circuit or signal processing circuitry tracks the ESAP signal metrics, the CPS, and physiological symptom resulting from the stimulation and the tracked information is displayed as a function or mapping of electrode position or distance from the CPS. In the example of FIG. 12, two mappings are shown, one for the physiologic symptom for pain and the other for digestion. For each mapping, ESAP signal amplitude is displayed with the electrode position from which it is recorded. The CPS is shown at the electrode position of the stimulation. The different functions and symptoms can be indicated by different shading or color. The electrode position of the CPS for one mapping may be a recording electrode for the other mapping.

The metric of the sensed ESAP signals may be normalized to a relative metric (e.g., signal amplitude may be normalized relative to a maximum signal amplitude determined at a given sensing time). Normalizing the metric may account for patient variability and within patient fluctuations (e.g., due to respiration, postural change, etc.). The mappings can include an indication of anatomical placement. For instance, the mappings in the example of FIG. 12 may show indications of the vertebra of the patient in relation to the electrodes. The mappings can be saved as function of rostrocaudal distance, electrode count, or other characteristic of the lead placement.

FIG. 13 is an illustration of another example of a user interface display presented by a control circuit of a neurostimulation device. The display 1300 includes a map of the locations of identified best ESAP signals versus physiologic symptoms associated with the best ESAP signals. The data for creating the map may be a collection of multiple ESAP signal maps from multiple patients that are combined to form a spinal dermatome-like map relating best ESAP signal location to physiologic symptoms. The mapping may designate vertebral levels (e.g., the T7, T8, and T9 vertebra in the example) where the best ESAP signals are expected for a given set of symptoms.

The user (e.g., a physician) may use the mapping as a guide for placement of the trial stimulation leads. The user may select one or more symptoms of interest. The symptom categories may be zones or bodily regions superimposed on the mapping and the user may select the symptom by selecting a region (e.g., using a computer mouse or touching a touchscreen). In certain examples, the categories are displayed checkbox list or drop-down menu and the user checks the appropriate box.

By checking a box or boxes, a dermatomal map or checklist can be referenced to determine which symptom target zones are shown. The symptom target zones in the display 1300 are shown symmetric and even for simplicity of the diagram, but the displayed zones may asymmetric and may be unilateral only (e.g., when the pain symptom is selected.

The targeting zones can be determined from the multiple signal maps based on localizing sensed ESAP signals (or other segmented signals), and from patient feedback on the neurostimulation (e.g., feedback of paresthesia-pain overlap). The mapping can be generated automatically. For example, a predetermined ESAP evoking algorithm can be performed by the neurostimulation device. The sensed ESAP signals evoked can be signal processed to identify and record locations of desired signal features (e.g., locations of maximum amplitude ESAP signals). The determined locations can then be mapped as shown in the display 1300. Optionally, the patient can provide feedback to validate symptom relief from the stimulation and the regions can be determined using the localized ESAP signals and the patient feedback. Additionally, the physician can provide feedback obtained through clinical testing.

The information presented to the user in FIGS. 9-13 can help the user determine the best ESAP signal for the patient during the trial stimulation. The best sensed ESAP signal can be stored, and the best ESAP signal may be stored as a best profile with one or both of the anatomical location of the best sensed ESAP signal and the physiologic effect correlated to the best sensed ESAP signal. The stored information can be used during the permanent procedure to guide placement of the permanent lead and provide automatic programming of the IPG to be connected to the permanent lead. The stored information can be used to adjust position of the permanent lead in the event of migration of the permanent lead to a changed position.

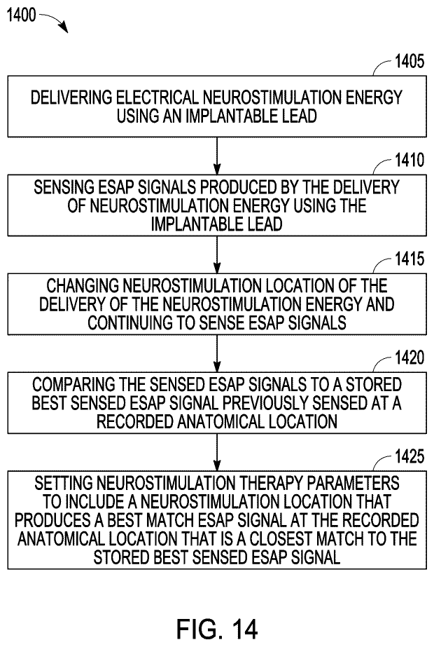

FIG. 14 is a flow diagram of an example of a method 1400 of operating a neurostimulation device (e.g., the example neurostimulation device 700 of FIG. 7). The neurostimulation device may be connected to a permanent implantable lead when performing the method 1400 to collect ESAP information as part of a permanent lead placement procedure. The neurostimulation device may be the same ETS used for the trial stimulation procedure or a different neurostimulation device. The collected ESAP information is compared to ESAP information from a trial implant procedure using a trial implantable lead different from the lead for permanent implant. The placement for the permanent lead is determined when the ESAP information produced with the permanent implantable lead matches the trial ESAP information. An initial placement of the permanent lead may be made using imaging.

At block 1405, the control circuit of the neurostimulation initiates delivery of neurostimulation therapy using the stimulation circuit and the permanent implantable lead to produce evoked potential signals in the patient. At block 1410, the control circuit initiates sensing of ESAP signals using the sensing circuit of the neurostimulation device and the permanent lead. The ESAP signals are produced by the delivery of neurostimulation energy.

At block 1415, the control circuit performs a process or algorithm that recurrently changes parameters of the neurostimulation including the anatomical location of the stimulation. The permanent lead includes multiple electrodes, and the control circuit changes the electrode or electrodes used to provide the neurostimulation. Electrodes not used for stimulation can be used to sense the ESAP signal response to the neurostimulation. For instance, the control circuit may perform a sweep of the electrodes of the trial lead in which most or all the available electrode stimulation combinations are used in turn for stimulation while other electrodes are used to sense the response to the stimulation.

At block 1420, the sensed ESAP signals are compared to the stored best sensed ESAP signal produced by the trial neurostimulation procedure. At block 1425, the lead placement and neurostimulation therapy parameters are set to a combination that produces the closest match to the stored best ESAP signal at the recorded anatomical location. In other words, the lead placement and neurostimulation therapy parameters are adjusted until permanent implant produces the results or nearly the results of the trial situation and placement.

During a permanent implant procedure, recordings can be taken at different CPS locations following a new prospective implant placement according to a pre-defined search pattern or search scheme defined by the user. The sensed ESAP signals resulting from each CPS may be recorded and measured on multiple electrodes for the placement at these sites. Profiles for the stimulation (e.g., ESAP signals recorded as function of rostrocaudal position of the lead, electrode number of the lead, CPS index, etc.) can be generated from recordings for each CPS. The generated profiles can be reviewed to find the closest match to the stored best profile shown to have a therapeutic effect during the trial stimulation procedure (e.g., via mathematical correlation).

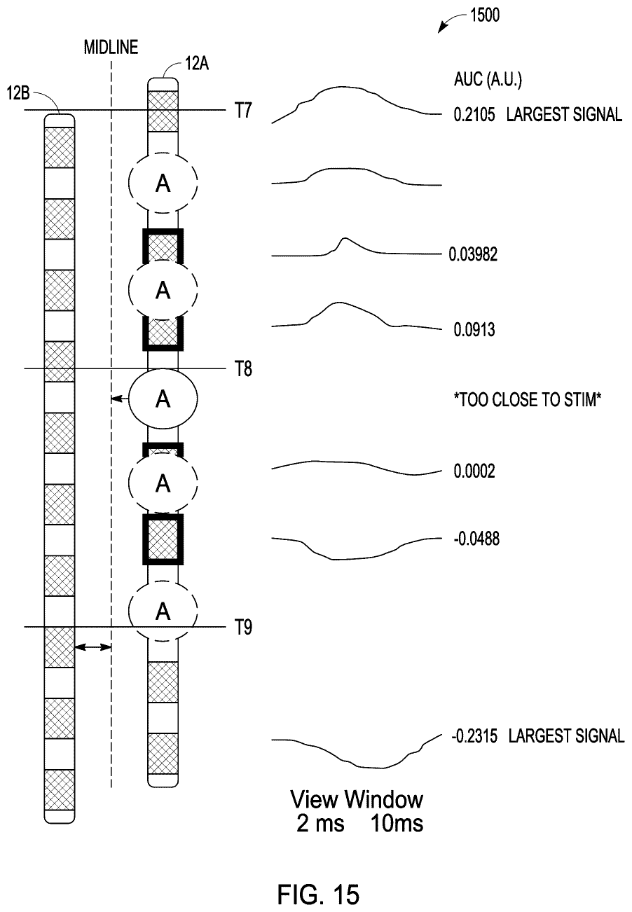

FIG. 15 is an illustration of another example of a user interface display that may be presented by the neurostimulation device during the permanent procedure. The display 1500 can be used to present the results of the neurostimulation and sensing using the permanent lead. The display 1500 includes a representation of two multielectrode implantable leads 12A, 12B. The position of the leads may correspond to a prospective lead placement for permanent leads. The five positions on lead 12A labeled “A” correspond to five CPS locations using different electrodes of the lead 12A. Four of the electrodes show the label “A” within a dashed circuit and on electrode shows the label “A” within a solid circle. The electrode with the solid circle represents a selection by the user to show the results of stimulation using that electrode. To the right of the lead 12A are waveforms of sensed ESAP signals recorded for the stimulation at the selected stimulation location. The waveforms are positioned to the right of the electrode used to sense the waveform.

The neurostimulation device may perform a feature versus location analysis to locate a match to the best result of the trial stimulation procedure. A feature versus location function over the entire lead may be used, or a narrower window may be established to improve results (e.g., to address stimulation electrodes closer to edges of lead and “aliasing” that might result). If therapy disturbances happen, the matching can be re-calibrated, use the match metric as a closed loop variable.

The display 1500 shows that the waveforms of the ESAP signals sensed at the top and bottom electrode have the largest amplitude. If one or both of the largest amplitude signals are the closest match to the stored best ESAP signal, the user may elect to use this prospective lead placement and the stimulation therapy setting for the permanent implant for the patient. The neurostimulation device may include an indication in the display 1500 of a close match to the stored best ESAP signal to one or more of the sensed ESAP signals. For example, signal processing circuitry of the neurostimulation device may calculate a correlation of matching of a sensed ESAP signal to the stored best sensed ESAP signal to determine the closest match. In some examples, the neurostimulation device performs a sweep of the stimulation using the available or selected stimulation electrodes and calculates a correlation to the stored best ESAP signal for all the resulting sensed ESAP signals. The neurostimulation device may include a recommendation to use the prospective lead placement as the permanent lead placement.

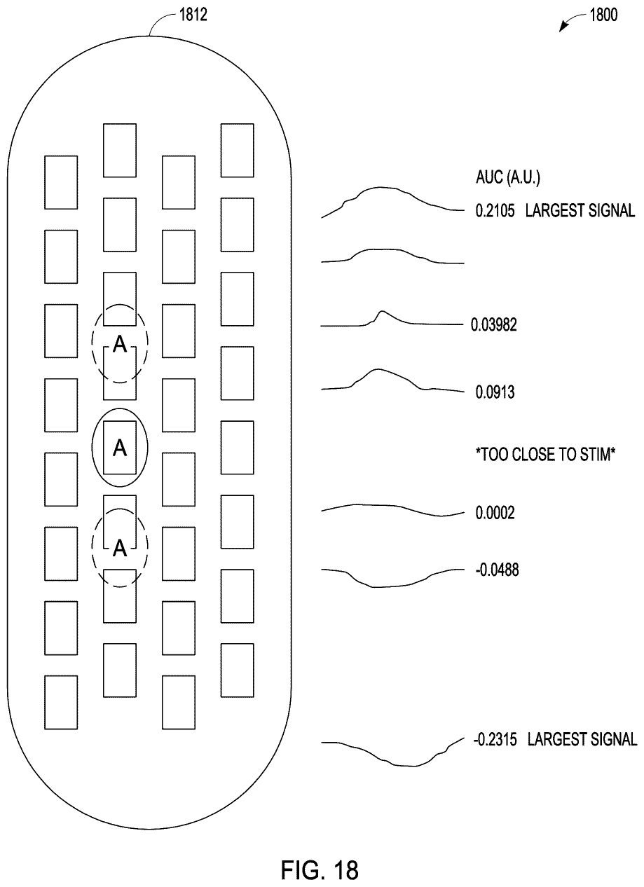

The trial lead or leads and the permanent lead or leads may be of different types. FIG. 18 is an illustration of another example of a user interface display that may be presented by the neurostimulation device during the permanent procedure. In the example of FIG. 18, the permanent lead 1812 is a paddle type lead. Similar to the example of FIG. 15, the display 1800 in FIG. 18 can be used to present the results of the neurostimulation and sensing using the permanent lead. The ESAP signals used to position the lead may have been sensed using the trial leads as in the example of FIG. 9. The neurostimulation device performs a sweep of the stimulation using the available or selected stimulation electrodes and calculates a correlation to the stored best ESAP signal for all the resulting sensed ESAP signals. Thus, the correlation is to the stored best ESAP signal even though the trial lead and the permanent are of different types.

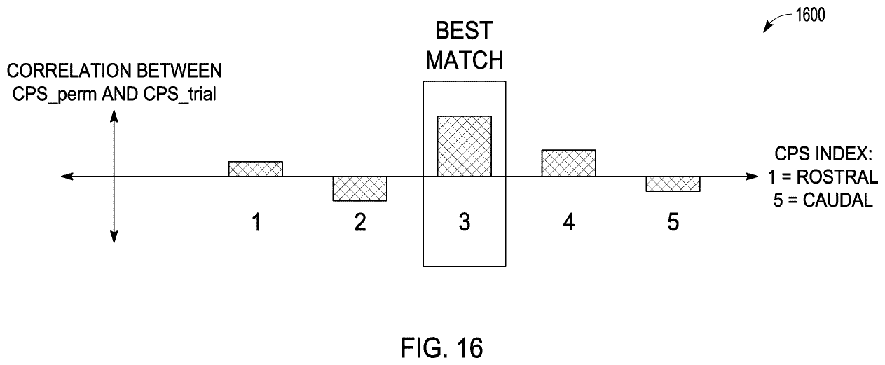

FIG. 16 is an illustration of another example of a user interface display that may be presented by the neurostimulation device during the permanent procedure. The display 1600 shows a horizontal representation of the electrodes of the permanent implantable lead with the vertical bars corresponding to recording electrode locations. The height of the bars corresponds to an ESAP signal metric of sensed ESAP signals resulting from the neurostimulation applied by the neurostimulation device during the permanent procedure.

The box around electrode “3” shows the best match between the CPS of the trial procedure and the CPS of the permanent procedure. The best match can be determined using various approaches such as maximum calculated correlation for the ESAP signal or metric, minimum squared error in the ESAP signal or metric, etc. The stimulation and location of the best match to the desired result should be selected for therapy at the time of permanent implant for the selected symptom.

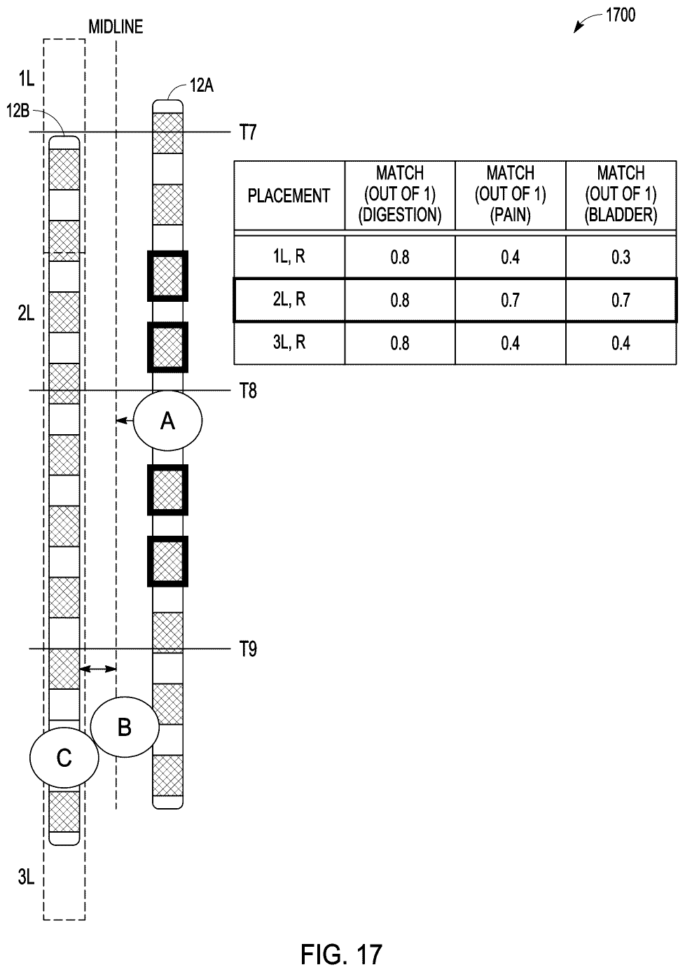

FIG. 17 is an illustration of another example of a user interface display that may be presented by the neurostimulation device during the permanent procedure. The neurostimulation device may perform multiple neurostimulation trials for multiple prospective lead placements and present the results using the display 1700. The display 1700 shows three CPS for three different therapeutic results, one for digestion (CPS A), pain (CPS B), and Bladder function (CPS C). The display 1700 also shows three lead placements in which the placement of lead 12B is changed relative to vertebra T7, T8, and T9.

Before or after symptom correlation is determined, a user may place permanent leads on the spinal cord using imaging (or an AI algorithm could be performed by the neurostimulation device to auto-test or auto-place leads), then stimulate using the programming carried over from the trial procedure. ESAP feature profile matches can be derived for each prospective lead placement, and the best or true placement can be recommended based on highest match obtained across multiple correlations. Three placements (1L, 2L, 3L) are shown in the display 1700, but more placements or fewer placements may be evaluated and displayed. Stimulation locations of interest may be translated into real electrode configurations via an algorithm performed by the neurostimulation device.

The best lead placement based on the central stimulation points can be highlighted and recommended. In the example of FIG. 17, the 2L placement is highlighted and recommended. The calculated correlation to the results for the three symptoms is presented. The placement stimulation and evaluation algorithm may be selected through a toggle button or switch displayed to the user. The best placement recommendation may be highlighted as in the example display 1700 or auto configured and displayed.

The stimulation algorithm can also fix the lead placement and test multiple CPS combinations in the same manner, (e.g., in the event there is major lead offset or lead migration). In certain examples, the lead placement can be defined based only on physiological response correlations and without need for vertebral locations or secondary imaging support. The stimulation and placement procedure may be performed without producing symptoms to gauge the placement as long as some effect of the stimulation can be used as a reference (e.g., a sensation produced at a location that is easily pinpointable location by the patient). Placements may be evaluated only through physiological symptoms without using anatomical or vertebral locations. The stimulation algorithm can be used at the time of permanent implant or to gauge the efficacy of the permanent placement during a later evaluation.

Stimulation lead placement for neurostimulation can be complicated and time consuming. The several embodiments described herein provide device-based assistance in lead placement. Evoking ESAP responses and using the location of the responses can provide information useful in guiding lead placement.

The embodiments described herein can be methods that are machine or computer-implemented at least in part. Some embodiments may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. Various embodiments are illustrated in the figures above. One or more features from one or more of these embodiments may be combined to form other embodiments.

The above detailed description is intended to be illustrative, and not restrictive. The scope of the disclosure should, therefore, be determined with references to the appended claims, along with the full scope of equivalents to which such claims are entitled.

What is claimed is:

1. A neurostimulation device, the device comprising:
a stimulation circuit configured to deliver electrical neurostimulation energy to a subject when coupled to an implantable lead;
a sensing circuit configured to sense evoked synaptic activity potential (ESAP) signals when coupled to the implantable lead; and
a control circuit operatively coupled to the stimulation circuit and the sensing circuit, and configured to:
initiate delivery of neurostimulation energy by the stimulation circuit;
initiate sensing by the sensing circuit of ESAP signals produced by the delivery of neurostimulation energy;
change at least one parameter of the neurostimulation energy and continue the sensing of the ESAP signals;
compare the sensed ESAP signals to a stored best sensed ESAP signal, wherein the best sensed ESAP signal was previously sensed at a recorded anatomical location; and
determine neurostimulation therapy parameters including a neurostimulation location to produce a best match ESAP signal at the recorded anatomical location that is a closest match to the stored best sensed ESAP signal.

2. The device of claim 1, wherein the control circuit is configured to:
recurrently change the neurostimulation location for multiple prospective lead placements of the implantable lead;
compare the sensed ESAP signals to the stored best sensed ESAP signal; and
determine, for each prospective lead placement, a neurostimulation location that produces the best match ESAP signal at the recorded anatomical location that is a closest match to the stored best sensed ESAP signal.

3. The device of claim 2, wherein the control circuit is configured to calculate a correlation of matching of the best match ESAP signal determined for each prospective lead placement to the stored best sensed ESAP signal.

4. The device of claim 2, wherein the control circuit is configured to:
change the neurostimulation location by changing combinations of electrodes of the implantable lead used to deliver the neurostimulation energy; and
change the combinations of electrodes of the implantable lead used to sense the ESAP signals in accordance with a change in the electrodes used to deliver the neurostimulation energy.

5. A computer-implemented method of operating a neurostimulation device to deliver electrical neurostimulation therapy when connected to an implantable lead, the method comprising:
delivering neurostimulation energy to produce evoked potential signals in a subject using a permanent implantable lead for permanent implant;
sensing evoked synaptic activity potential (ESAP) signals using the permanent implantable lead;
changing at least one parameter of the neurostimulation energy and continuing to sense the ESAP signals;
comparing the sensed ESAP signals to a stored best sensed ESAP signal previously sensed at a recorded anatomical location; and
setting neurostimulation therapy parameters to include a neurostimulation location that produces a best match ESAP signal at the recorded anatomical location that is a closest match to the stored best sensed ESAP signal.

6. The method of claim 5, including:
recurrently changing the neurostimulation and comparing the sensed ESAP signals to a stored best sensed ESAP signal for multiple prospective lead placements of the permanent implantable lead; and
determining, for each prospective lead placement, a neurostimulation location that produces the best match ESAP signal at the recorded anatomical location that is a closest match to the stored best sensed ESAP signal.

7. The method of claim 6, including calculating a correlation of matching of the best match ESAP signal determined for each prospective lead placement to the stored best sensed ESAP signal.

8. The method of claim 5, including:
changing an anatomical location of delivering the neurostimulation energy by changing combinations of electrodes of the permanent implantable lead used to provide the neurostimulation; and
changing location of the sensed ESAP signals by changing the combinations of electrodes of the permanent implantable lead used to sense the ESAP signals.

9. The method of claim 5,
wherein the stored best sensed ESAP signal includes a recording of the best sensed ESAP signal, the anatomical location of the best sensed ESAP signal, and a physiological effect of the trial neurostimulation correlated to the best sensed ESAP signal.

10. The method of claim 9, including:
delivering trial neurostimulation using a stimulation electrode of a trial implantable lead;
sensing trial ESAP signals using multiple recording electrode combinations of the trial implantable lead different from the stimulation electrode combination;
displaying a representation of the stimulation electrode and the recording electrodes;
displaying a metric of the sensed trial ESAP signals in association with the displayed recording electrodes; and
receiving a selection of the best sensed ESAP signal by a user interface of the neurostimulation device.

11. The method of claim 10, including displaying an indication of anatomical location with the representation of the stimulation electrode and the recording electrodes.

12. The method of claim 9, including:
delivering trial neurostimulation using a stimulation electrode of a trial implantable lead;
sensing trial ESAP signals using multiple recording electrodes of the trial implantable lead different from the stimulation electrode;
displaying a representation of the stimulation electrode, the recording electrodes, and a representation of one or more physiologic effects in relation to the stimulation electrode and recording electrodes; and
receiving a selection of the best sensed ESAP signal by a user interface of the neurostimulation device.

13. The method of claim 9, including:
delivering trial neurostimulation using a stimulation electrode of a trial implantable lead;
sensing trial ESAP signals using multiple recording electrodes of the trial implantable lead different from the stimulation electrode;
displaying a metric of the sensed trial ESAP signals in association with lead position of a corresponding recording electrode for a sensed trial ESAP signal;
displaying the lead position of the stimulation electrode; and
receiving a selection of the best sensed ESAP signal by a user interface of the neurostimulation device.

14. The method of claim 9, including:
displaying a representation of anatomical locations available for producing and sensing the trial ESAP signals; and
displaying regions of physiologic symptoms in association with the anatomical locations.

15. A neurostimulation device, the device comprising:
a stimulation circuit configured to deliver electrical neurostimulation energy to a subject when coupled to an implantable lead;
a sensing circuit configured to sense evoked synaptic activity potential (ESAP) signals when coupled to the implantable lead; and
a control circuit operatively coupled to the stimulation circuit and the sensing circuit, and configured to:
initiate delivery of neurostimulation energy by the stimulation circuit;
initiate sensing, by the sensing circuit, of ESAP signals produced by the neurostimulation energy;
recurrently change neurostimulation location of the delivery of the neurostimulation energy;
identify a best sensed ESAP signal determined according to a physiologic effect produced by the neurostimulation energy; and
store the best sensed ESAP signal, an anatomical location of sensing of the best sensed ESAP signal, the physiologic effect and the stimulation location associated with the physiological effect.

16. The device of claim 15, including:
a user interface including a display;
wherein the control circuit is configured to:
initiate delivery of trial neurostimulation using a stimulation electrode of the implantable lead;
initiate sensing of the ESAP signals using multiple recording electrodes of the trial implantable lead different from the stimulation electrode;
present, using the display, a representation of the stimulation electrode and the recording electrodes;
present a metric of the sensed trial ESAP signals in association with the recording electrodes; and
receive a selection of the best sensed ESAP signal via the user interface.

17. The device of claim 16, wherein the control circuit is configured to display an indication of anatomical location with the representation of the stimulation electrode and the recording electrodes.

18. The device of claim 15, including:
a user interface including a display and a user input;
wherein the control circuit is configured to:
initiate delivery of trial neurostimulation using a stimulation electrode of the implantable lead;
initiate sensing of the ESAP signals using multiple recording electrodes of the trial implantable lead different from the stimulation electrode; and
present, using the display, a representation of the stimulation electrode, the recording electrodes, and a representation of one or more physiologic effects in relation to the stimulation electrode and recording electrodes.

19. The device of claim 15, including:
a user interface including a display;
wherein the control circuit is configured to:
initiate delivery of trial neurostimulation using a stimulation electrode of the implantable lead;
initiate sensing of the ESAP signals using multiple recording electrodes of the trial implantable lead different from the stimulation electrode;
present, using the display, a metric of the sensed ESAP signals in association with position of one or more of the implantable lead, a stimulation anode, a stimulation cathode, inactive electrodes, a central point of stimulation, and electrode fractionalization.

20. The device of claim 15, including:
a user interface including a display; and
wherein the control circuit is configured to:
present, using the display, a representation of anatomical locations available for producing and sensing the ESAP signals; and
present regions of physiologic symptoms in association with the anatomical locations.
